(19) United States
(12) Patent Application Publication
(10) Pub. No.: US 2018/0293663 A1
(43) Pub. Date: Oct. 11, 2018

(54) SELECTION OF A SPECIALIST PHYSICIAN FOR INCLUSION IN A NARROW-NETWORK HEALTHCARE PLAN

(71) Applicant: Cedars-Sinai Medical Center, Los Angeles, CA (US)

(72) Inventors: Brennan Spiegel, Los Angeles, CA (US); Garth Fuller, Los Angeles, CA (US)

(73) Assignee: Cedars-Sinai Medical Center, Los Angeles, CA (US)

(21) Appl. No.: 15/947,230

(22) Filed: Apr. 6, 2018

Related U.S. Application Data
(60) Provisional application No. 62/482,903, filed on Apr. 7, 2017.

(51) Int. Cl. G06Q 40/08; G16H 50/70

(52) U.S. Cl. CPC G06Q 40/08; G16H 50/70

(57) ABSTRACT

Aspects of the present disclosure relate to determining which specialist physician to include within narrow-network healthcare plan. The determination can involve generating a composite score for specialist physician performance. The composite score can be based on an objective component or score and a subjective component or score. The objective score can be generated based on processing electronic health record data applicable to the specialist physician and patients of the physician. The subjective score can be based on surveys of participants with an understanding of the specialist physician skill as a physician.

### CROSS-REFERENCE TO RELATED APPLICATION

The present application claims the benefit of and priority to U.S. Provisional Application No. 62/482,903, filed Apr. 7, 2017, and entitled “SELECTION OF A SPECIALIST PHYSICIAN FOR INCLUSION IN A NARROW-NETWORK HEALTHCARE PLAN,” the contents of which are hereby incorporated by reference herein in their entirety.

### TECHNICAL FIELD

The present invention relates to selecting individuals to include within a narrow-network healthcare plan. More particularly, the present invention relates to devices, systems, and methods for selecting high-performing, low-cost healthcare providers (e.g., physicians, and specialist physicians) to include within a narrow-network healthcare plan.

### BACKGROUND

Increasingly, healthcare insurers control costs by selectively contracting with high-performing, low-cost providers through narrow-network healthcare plans. Narrow-network healthcare plans cover services via fewer physicians in a geographic area than do standard plans and position insurers to act as informed and invested intermediaries in the healthcare process. Through narrow-networks, insurers seek to control costs by selectively contracting with high-performing, low-cost providers and healthcare systems. Healthcare entities are faced with selecting physicians—particularly specialist physicians—for inclusion in these narrow-network healthcare plans. There is a pressing need to develop methods to transparently and effectively determine specialist physicians for these plans.

Performance measurement at the physician level is not straightforward, however, and has emerged as a particularly thorny topic in healthcare. Limitations in available data—as well as conceptual difficulties in measuring quality and value in healthcare broadly—make specialist physician performance assessment difficult from both statistical and organizational perspectives. The reliability of performance measures is often low due to small sample sizes, heterogeneity in patient populations, and the presence of measurement error. Additional threats to reliability and validity include patient- and procedure-attribution problems, the need for case-mix adjustment, and the presence of data-collection errors.

According to the foregoing issues, a need exists that addresses and mitigates these challenges by using a mixed-methods process that achieves important measurement, selection, and organizational goals while also promoting feelings of organizational justice among physicians.

### SUMMARY

An aspect of the present disclosure includes a method for including one or more specialist physicians in a narrow-network healthcare plan. The method includes determining a subset of electronic health record data corresponding to the one or more specialist physicians. The one or more specialist physicians are a subset of physicians associated with a healthcare entity based on a type of specialty. The subset of the electronic health record data is based on the type of specialty. The method also includes applying a weighting to the subset of the electronic health record data, where the weighting is tailored to the type of specialty. The method also includes processing the weighted subset of the electronic health record data to generate a performance score for each specialist physician of the one or more specialist physicians. Further, the method includes processing a plurality of peer reviews completed by participants associated with the healthcare entity concerning the one or more specialist physicians to generate a reputation score for each specialist physician of the one or more specialist physicians. The reputation score quantifies reputations of the one or more specialist physicians among the participants associated with the healthcare entity. The method also includes processing the performance score and the reputation score for each specialist physician of the plurality of specialist physicians to determine a composite score for selecting the one or more specialist physicians to include in the narrow-network healthcare plan.

Another aspect of the present disclosure includes a method of modifying a peer review of an individual to appear quantitative without changing an outcome of the peer review. The method includes conducting the peer review of the individual. The individual can be a specialist physician at a healthcare entity. The peer review can include surveying a plurality of participants related to the healthcare entity for opinions of the plurality of participants related to the individual. The method also includes processing the peer review to generate a reputation score for the individual, and processing electronic health record data corresponding to treatment of a plurality of patients by the individual to generate a performance score. The performance score can quantify a quality of care, an efficiency of care, or a combination of quality and efficiency of care provided to the plurality of patients by the individual. The method further includes processing the performance score and the reputation score to determine a composite score applicable to the individual and for determining whether to include the individual within a narrow-network healthcare plan. The reputation score can be weighted relative to the performance score such that the determination of whether to include the individual within the narrow-network healthcare plan depends on only the reputation score.

Another aspect of the present disclosure includes a system for including one or more specialist physicians in a narrow-network healthcare plan. The system includes one or more databases storing electronic health record data and logic circuitry. The logic circuitry is configured to determine a subset of the electronic health record data corresponding to the one or more specialist physicians. The one or more specialist physicians is a subset of physicians associated with a healthcare entity based on a type of specialty, and the subset of the electronic health record data is based on the type of specialty. The logic circuitry is further configured to apply a weighting to the subset of the electronic health record data, the weighting being based on the type of specialty. The logic circuitry is further configured to generate a performance score for each specialist physician of the one or more specialist physicians based on the weighted subset of the electronic health record data. The logic circuitry is further configured to generate a reputation score for each specialist physician of the one or more specialist physicians based on a plurality of peer reviews completed by participants associated with the healthcare entity concerning the one or more specialist physicians. The reputation score quantifies reputations of the one or more specialist physicians among the participants associated with the healthcare entity. The logic circuitry is further configured to determine a composite score for selecting the one or more specialist physicians to include in the narrow-network healthcare plan based on the performance score and the reputation score for each specialist physician of the plurality of specialist physicians.

Another aspect of the present disclosure includes a system for modifying a peer review of an individual to appear quantitative without changing an outcome of the peer review. The system includes a terminal for receiving the peer review. The peer review includes a survey of a plurality of participants related to the healthcare entity for opinions of the plurality of participants related to the individual. The system further includes logic circuitry. The logic circuitry is configured to generate a reputation score for the individual based on the peer review. The logic circuitry is further configured to generate a performance score based on electronic health record data corresponding to treatment of a plurality of patients by the individual, where the performance score quantifies a quality of care, an efficiency of care, or a combination thereof provided to the plurality of patients by the individual. The logic circuitry is further configured to determine a composite score applicable to the individual based on the performance score and the reputation score for determining whether to include the individual within a narrow-network healthcare plan. The reputation score is weighted relative to the performance score such that the determination of whether to include the individual within the narrow-network healthcare plan depends on only the reputation score.

Another aspect of the present disclosure includes an apparatus for including one or more specialist physicians in a narrow-network healthcare plan. The apparatus includes a processor and a memory including computer program code for one or more programs. The memory and the computer program code are configured to, with the processor, cause the apparatus to determine a subset of electronic health record data corresponding to the one or more specialist physicians. The one or more specialist physicians are a subset of physicians associated with a healthcare entity based on a type of specialty, and the subset of the electronic health record data is based on the type of specialty. The memory and the computer program code further are configured to cause the apparatus to apply a weighting to the subset of the electronic health record data, with the weighting being tailored to the type of specialty. The memory and the computer program code further are configured to cause the apparatus to generate a performance score for each specialist physician of the one or more specialist physicians based on the weighted subset of the electronic health record data. The memory and the computer program code further are configured to cause the apparatus to generate a reputation score for each specialist physician of the one or more specialist physicians based on a plurality of peer reviews completed by participants associated with the healthcare entity concerning the one or more specialist physicians. The reputation score quantifies reputations of the one or more specialist physicians among the participants associated with the healthcare entity. The memory and the computer program code further are configured to cause the apparatus to determine a composite score for selecting the one or more specialist physicians to include in the narrow-network healthcare plan based on the performance score and the reputation score for each specialist physician of the plurality of specialist physicians.

Another aspect of the present disclosure includes an apparatus for modifying a peer review of an individual to appear quantitative without changing an outcome of the peer review. The apparatus includes a processor and a memory including computer program code for one or more programs. The memory and the computer program code are configured to, with the processor, cause the apparatus to conduct the peer review of the individual. The individual is a specialist physician at a healthcare entity, and the peer review includes surveying a plurality of participants related to the healthcare entity for opinions of the plurality of participants related to the individual. The memory and the computer program code further are configured to cause the apparatus to process the peer review to generate a reputation score for the individual, and process electronic health record data corresponding to treatment of a plurality of patients by the individual to generate a performance score. The performance score quantifies a quality of care, an efficiency of care, or a combination thereof provided to the plurality of patients by the individual. The memory and the computer program code further are configured to cause the apparatus to process the performance score and the reputation score to determine a composite score applicable to the individual and for determining whether to include the individual within a narrow-network healthcare plan. The reputation score is weighted relative to the performance score such that the determination of whether to include the individual within the narrow-network healthcare plan depends on only the reputation score.

Aspects of the present disclosure also include one or more devices, one or more systems, and/or combinations thereof that can perform any of the methods discussed herein.

These and other capabilities of the disclosed devices, systems, and methods will be more fully understood after a review of the following figures, detailed description, and claims.

### BRIEF DESCRIPTION OF THE DRAWINGS

FIG. 1 is a diagram of a system capable of selecting specialists to include within a narrow-network health care plan, in accord with aspects of the present disclosure.

FIG. 2 is a flowchart of a process for including one or more specialist physicians within a narrow-network healthcare plan, in accord with aspects of the present disclosure.

FIG. 3 is a flowchart of a process for modifying a peer review of an individual to appear quantitative without changing an outcome of the peer review, in accord with aspects of the present disclosure.

FIG. 4 is a diagram of hardware that can be used to implement aspects of the present disclosure.

FIG. 5 is a diagram of a chip set that can be used to implement aspects of the present disclosure.

While the devices, systems, and methods discussed herein are susceptible to various modifications and alternative forms, specific embodiments have been shown by way of example in the drawings and will be described in detail herein. It should be understood, however, that the description is not intended to be limited to the particular forms disclosed. Rather, the description is to cover all modifications, equivalents, and alternatives falling within the spirit and scope of the present disclosure as defined by the appended claims.

### DETAILED DESCRIPTION

There is shown in the drawings and will herein be described in detail one or more embodiments of the device, system, and method with the understanding that the present disclosure is to be considered as an exemplification of the principles of the disclosed herein and is not intended to limit the broad aspects solely to the embodiments illustrated. For purposes of the present detailed description, the singular includes the plural and vice versa (unless specifically disclaimed); the word “or” shall be both conjunctive and disjunctive; the word “all” means “any and all”; the word “any” means “any and all”; and the word “including” means “including without limitation.” Additionally, the singular terms “a,” “an,” and “the” include plural referents unless context clearly indicates otherwise.

The determination of which specialist to include within a narrow-network healthcare plan involves determining a composite score for physician performance. The composite score can be based on an objective component or score and a subjective component or score. The objective score can be based on electronic health record data, and the subjective score can be based on the reputation of the physician as indicated by survey responses.

The objective score can be based on analyzing metrics stored as electronic health record data. The electronic health record data can quantify the quality of care, the efficiency of care, or a combination of the quality and efficiency of care provided by a physician, such as a specialist physician. The electronic health record data provides an objective view on the specialist physician's care or treatment of patients. Therefore, in some aspects, the objective score can be referred to or considered a performance score.

Exemplary metrics included within the electronic health record data include (1) choosing wisely/best practice alerts; (2) length of patient stay; (3) 30-day readmission rate; (4) severity-adjusted total cost of care; (5) generic drug utilization rate; and (6) office visit patient satisfaction. However, any type of metric logged by a healthcare provider, such as a hospital, that is related to a physician's care of a patient can be used as the electronic health record data. Not all of the metrics collected must be used to calculate a performance score. Additionally, not all of the physicians may have data for all of the metrics, both of which are discussed further below.

In some aspects, the specific metrics used to calculate the performance score can vary depending on the specialty of the specialist physician being analyzed. Table 1 includes exemplary metrics used for exemplary specialties.

TABLE 1
Metrics grouped by specialty.

| Specialty | Metric Label |
|---|---|
| Cardiology | Choosing Wisely/Best Practice Alerts |
| | Length of Stay |
| | 30-day Readmission Rate |
| | Severity-Adjusted Total Cost of Care |
| | Generic Drug Utilization Rate |
| ENT | Choosing Wisely/Best Practice Alerts |
| | Severity-Adjusted Total Cost of Care |
| | Generic Drug Utilization Rate |
| | Length of Stay |
| Endocrinology | Average Number Fine-Needle Aspirations Per |
| | Year Per Patient with Thyroid Nodule |
| | Choosing Wisely/Best Practice Alerts |
| | Severity-Adjusted Total Cost of Care |
| | Generic Drug Utilization Rate |
| | Length of Stay |
| Gastroenterology | Percentage of Outpatients Who Undergo Both Upper |
| | Endoscopy and Colonoscopy in a Single Session |
| | Percentage of outpatient abdominal pain consults |
| | that result in a CT or MRI |
| | Choosing Wisely/Best Practice Alerts |
| | Severity-Adjusted Total Cost of Care |
| | Generic Drug Utilization Rate |
| General Surgery | Length of Stay |
| | 30-day Readmission Rate |
| | Severity-Adjusted Total Cost of Care |
| | Generic Drug Utilization Rate |
| Nephrology | Percentage of dialysis sessions for which albumin is |
| | ordered Percentage of inpatient consults |
| | for which an Erythropoietin Stimulating Agent ESA |
| | is ordered - measured by patient days |
| | Choosing Wisely/Best Practice Alerts |
| | Severity-Adjusted Total Cost of Care |
| | Generic Drug Utilization Rate |
| | Length of Stay |
| Neurology | Number of headache cases for which outpatient |
| | CT or MRI is performed |
| | Number of stroke or TIA (denominator) cases for |
| | which CTA or MRA (numerator) is performed |
| | Choosing Wisely/Best Practice Alerts |
| | Severity-Adjusted Total Cost of Care |
| | Generic Drug Utilization Rate |
| | Length of Stay |
| OB/GYN | Percentage of vaginal deliveries resulting in |
| | episiotomy Rates of C-Section |
| | Severity-Adjusted Total Cost of Care |
| | Generic Drug Utilization Rate |
| | Office visit patient Satisfaction |
| | Length of Stay |

In some aspects, each metric is assigned a weight. The weight of the metric quantifies how important or significant that metric is at representing the performance of the specialist physician, particularly in comparison to the other metrics. Some metrics can be more important/significant for one specialty than for another specialty. Thus, in some aspects, each metric can be assigned a different weight depending on the specialty in consideration. For example, the metric of length of stay can be assigned a different weight for cardiologists as compared to neurologists. Table 2 lists the metrics separated by specialty from Table 1, along with the assigned weights.

TABLE 2
Metrics with weights.

| | | Scoring |
|---|---|---|
| Specialty | Metric Label | Weights |
| Cardiology | Choosing Wisely/Best Practice Alerts | 50 |
| | Length of Stay | 10 |
| | 30-day Readmission Rate | 5 |
| | Severity-Adjusted Total Cost of Care | 70 |
| | Generic Drug Utilization Rate | 20 |
| ENT | Choosing Wisely/Best Practice Alerts | 40 |
| | Severity-Adjusted Total Cost of Care | 50 |
| | Generic Drug Utilization Rate | 30 |
| | Length of Stay | 30 |
| Endocrinology | Average Number Fine-Needle Aspirations Per Year Per | 60 |
| | Patient with Thyroid Nodule | |
| | Choosing Wisely/Best Practice Alerts | 50 |
| | Severity-Adjusted Total Cost of Care | 70 |
| | Generic Drug Utilization Rate | 60 |
| | Length of Stay | 10 |
| Gastroenterology | Percentage of Outpatients Who Undergo Both Upper | 60 |
| | Endoscopy and Colonoscopy in a Single Session | |
| | Percentage of outpatient abdominal pain consults that | 30 |
| | result in a CT or MRI | |
| | Choosing Wisely/Best Practice Alerts | 50 |
| | Severity-Adjusted Total Cost of Care | 70 |
| | Generic Drug Utilization Rate | 20 |
| General Surgery | Length of Stay | 50 |
| | 30-day Readmission Rate | 50 |
| | Severity-Adjusted Total Cost of Care | 20 |
| | Generic Drug Utilization Rate | 10 |
| Nephrology | Percentage of dialysis sessions for which albumin is | 30 |
| | ordered | |
| | Percentage of inpatient consults for which an | 30 |
| | Erythropoietin Stimulating Agent ESA is ordered - | |
| | measured by patient days | |
| | Choosing Wisely/Best Practice Alerts | 50 |
| | Severity-Adjusted Total Cost of Care | 70 |
| | Generic Drug Utilization Rate | 10 |
| | Length of Stay | 20 |
| Neurology | Number of headache cases for which outpatient CT or MRI | 30 |
| | is performed | |
| | Number of stroke or TIA (denominator) cases for which | 30 |
| | CTA or MRA (numerator) is performed | |
| | Choosing Wisely/Best Practice Alerts | 50 |
| | Severity-Adjusted Total Cost of Care | 70 |
| | Generic Drug Utilization Rate | 40 |
| | Length of Stay | 10 |
| OB/GYN | Percentage of vaginal deliveries resulting in episiotomy | 30 |
| | Rates of C-Section | 60 |
| | Severity-Adjusted Total Cost of Care | 50 |
| | Generic Drug Utilization Rate | 10 |
| | Office visit patient Satisfaction | 70 |
| | Length of Stay | 30 |

The weights of the metrics can be determined according to various approaches. According to one approach, the weights are determined based on surveys completed by physicians. Responses from physicians on the importance of each metric can be used to generate the weights. The surveys can be for assigning metric weights applied to a specific specialty but completed by physicians across all specialties, including general physicians. Alternatively, the surveys can be for assigning metric weights to a specific specialty but completed by only specialist physicians within that specialty.

According to one aspect, the surveys can be based on a RAND appropriateness rating survey. Participants of the survey can be asked to rank the importance of a metric in demonstrating the performance of a physician. Following the RAND rating style, responses can be provided on a scale, such as on a scale of 0 to 10, 1 to 9, etc., where one end of the scale represents the highest level of importance and the opposite end of the scale represents the lowest level of importance. The combined scores from the surveys across the physicians can then be used to generate a weight for that particular metric. For example, the combined scores can be averaged, summed, normalized, and the like, including combinations thereof, to generate the weight for that particular metric.

Each metric can initially be in the form of raw data. For example, the metric of a generic drug utilization rate can be in the form of a percentage, such as the percentage of time a physician prescribes a generic drug as compared to a non-generic drug. The metric in the raw form for a particular specialist can be standardized. In some aspects, standardization can be used to facilitate ease of understanding or interpretation for physicians.

One method of standardization involves a two-step process. The first step of the process concerns determining a z-score for the metric as applied to a particular specialist. Equation 1 below represents the calculation of a z-score:

$${z}_{X ( i )} = \frac{{X}_{i} - M}{{s}_{X}} \qquad ( 1 )$$

where zₓ₍ᵢ₎ is the z-score for the metric x pertaining to the specialist i, xᵢ is the metric value for the specialist i, M is the arithmetic mean of the metric x based on raw values of the metric x, and s_{X} is the standard deviation for the metric x. In some aspects, M can be the arithmetic mean of the metric x based on the raw values of the metric x for all physicians. Alternatively, M can be the arithmetic mean of the metric x based on the raw values of the metric x for only physicians of the specialty of the specialist physician for which the performance score is being determined.

The second step of the process concerns assigning a percentage of the weight for the particular metric based on the calculated value of the z-score. Table 3 below is a relational table used to assign a percentage of the weight for the metric based on the z-score.

TABLE 3
Relational table of z-scores to metric weights.

If zₓ₍ᵢ₎ ≥ 2, assign 100% of metric weight
If 2 > zₓ₍ᵢ₎ ≥ 1, assign 95% of metric weight
If 1 > zₓ₍ᵢ₎ ≥ 0, assign 85% of metric weight
If 0 > zₓ₍ᵢ₎ ≥ −1, assign 75% of metric weight
If −1 > zₓ₍ᵢ₎ ≥ −2, assign 65% of metric weight
If −2 > zₓ₍ᵢ₎, assign 55% of metric weight

After standardization of all of the metrics for a particular specialist, the standardized values can be summed to yield a performance score for the specialist. For missing data for a particular metric, the metric can be dropped from the calculation of the final score. During calculation of the final composite score, the dropped metrics can be accounted for to not limit specialist physicians that do not have certain metrics, as described further below.

The subjective score is based on reviews of the specialist physicians by peers. In some aspects, the peers can be only physicians, such as other physicians within the same specialty, within a different specialty, or non-specialist physicians, such as general care or primary care physicians. In some aspects, the peers can include or alternatively be stakeholders of the healthcare facility, such as non-physician administrators and/or management that are familiar with the performance of physicians. In some examples, the stakeholders can include individuals from departments that interact with the specialist physicians (e.g., nursing, pharmacy, etc.), directors or administrators of medicine programs (e.g., hospitalist medicine department, emergency department, intensive care unit, etc.), and personnel with broad knowledge of specialist performance (e.g., chief of medical staff, academic dean, palliative care consultants, etc.). In some aspects, the participants reviewed can be specific to the specialty. For example, dialysis staff can be included for nephrologists, endoscopy staff can be included for gastroenterologists, and anesthesiologists can be included for surgical specialists, among others.

The subjective score can be based on asking the participants to rate each specialist physician with whom they are familiar. The rating can be based on a single question or based on multiple questions. In some aspects, the rating for each question asked can be based on a 9-point modified RAND scale ranging from 1 (“completely disagree”) to 9 (“completely agree”).

In some aspects, the rating can be based on a single question or statement, such as to provide a number from 1 (“completely disagree”) to 9 (“completely agree”) in response to the following statement: “Dr. [Name] is a strong partner in value-based healthcare.” However, more than one question or statement can be used to gather responses from the participants. The results of the survey can then be averaged for each specialist physician to generate the subjective score for the specialist physician.

The objective and subjective scores are then combined to generate the composite score for the specialist physician. In some aspects, the composite score can then be calculated according to Equation 2:

Quantₜ+Qualₛ (2)

where Quantₜ is the total performance score (e.g., quantitative score) of the specialist physician and Qualₛ is the subjective score of the specialist physician. Thus, the raw scores can be summed to obtain the composite score.

In some aspects, the composite score can be calculated in such a way as to allow the subjective score to account for missing or low-sample size electronic health record data in the quantitative score. For example, the subjective score can be used to increase the composite score for physicians with dropped metrics by using Equation 3 to calculate the composite score:

Quantₜ+(Quantₘ−Quantᵢ)−Qualₚ+Qualₛ (3)

where Quantₘ is the maximum possible performance score (i.e., sum of metric weights), Quantᵢ is the total possible performance score for the specialist physician (i.e., the sum of the metric weights excluding any dropped metrics), and Qualₚ is the specialist physician's peer survey mean percentage (i.e., the percentage of the subjective score relative to the total possible subject score). Thus, the missing data is accounted for by providing the data based on the assumption that the data would reflect the specialist physician's reputation/subjective score.

In some aspects, the weighting of the subjective score relative to the performance score can be modified. In combination with the modification of the performance score for dropped metrics above, the composite score can be calculated according to Equation 4 to adjust the weighting:

Quantₜ+(Quantₘ−Quantᵢ)−Qualₚ+Qualₚ−Qualₘ (4)

where Qualₘ is the maximum possible qualitative score, such as the sum of the metric weights. When Qualₘ is the sum of the metric weights, the performance score is weighted equally as the subjective score. Alternatively, Qualₘ can be something besides the sum of the metric weights, such as some value higher or lower than the sum of the metric weights. As a value higher than the sum of the metric weights, the subjective score can be weighted higher than the performance score. As a value lower than the sum of the metric weights, the subjective score can be weighted lower than the performance score.

In some aspects, the objective score, the subjective score, or both can be modified based on other criteria and/or weighting. In some aspects, the experience, such as the number of years the specialist physician has been practicing, can be used as a weighting, such as by providing a greater weight to the electronic health data based on a larger number of years of practicing by the specialist physician.

In some aspects, the performance score generally agrees with the subjective score. In other words, the reputation that a specialist physician has among his or her peers accurately reflects the electronic health record data that applies to the specialist physician. For example, a specialist physician can have a low reputation among his or her peers in part because the peers are aware of the specialist physician's performance related to patient care.

Further, in some aspects, it may be easier (e.g., less expensive for data collection, storage, securement, etc.) to determine which specialists to be included within a narrow network based solely on a subjective score. A subjective score merely requires completion of surveys by participants. In contrast, data collection and application of the data to the determination of a specialist physician to include within a narrow network may be expensive.

However, a determination that affects a specialist physician that is based solely on an apparent popularity contest among the specialist physician's peers may cause issues with the specialist physician. Additionally, in some aspects, participants of the survey may adjust their evaluation of a physician to be less accurate if the participants believe that their rating of the specialist physician may be provided to or otherwise discovered by the specialist physician.

Thus, in some aspects, the determination of which specialist to include within a narrow-network healthcare plan can be determined based only on the subjective score. However, “noise” can be combined with the subjective score to provide the appearance of a fair (e.g., non-biased or popularity contest) evaluation of which specialist to include within the narrow-network healthcare plan. The “noise” can be analysis of the electronic health record data and the generated performance score. However, because the performance score generally agrees with the subjective score, the performance score can be considered “noise” that is entered into the determination of which specialist physician to include because the performance score does not affect the ultimate determination of the composite score. Alternatively, or in addition, the subjective score can be weighted relative to the performance score such that, even with a difference in the outcome of a performance score versus a subjective score relative to the peers, the performance score does not affect the outcome of which specialist to include based on the composite score.

In some aspects, a determination of composite scores for multiple physicians can be made based on equal weighting of the performance score and the subjective score for each physician. A second determination of composite scores for the physicians can be made using only the subjective scores. If the outcomes of the two determinations vary, such as one determination resulting in different specialist physicians to include in narrow network than the other, the weighting of the performance score versus the subjective score can be modified to cause the determination of the composite score based on the performance score and the subjective score to have the same result.

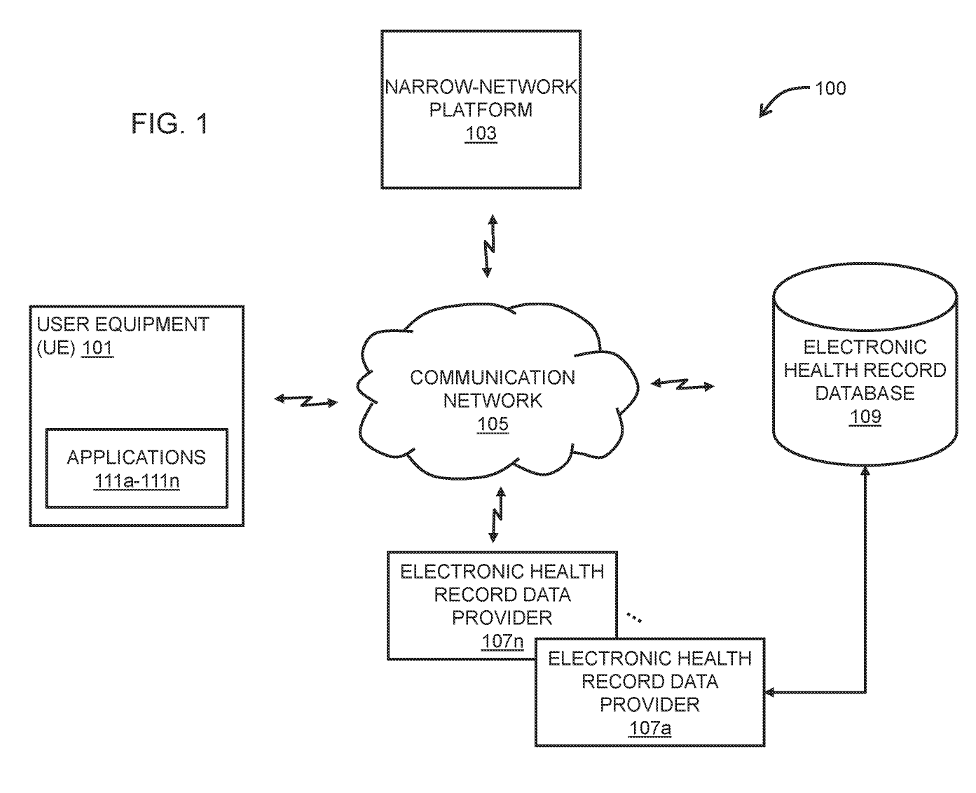

FIG. 1 illustrates a system 100 that can be used to select specialist physicians to include within a narrow-network healthcare plan, in accord with aspects of the present disclosure. The system 100 comprises a user equipment (UE) 101 having connectivity to a narrow-network platform 103 via a communication network 105. By way of example, the communication network 105 of system 100 includes one or more networks such as a data network, a wireless network, a telephony network, or any combination thereof.

The UE 101 can be any type of computing device, such as a desktop computer, a laptop computer, a notebook computer, a netbook computer, a tablet computer, a personal communication system (PCS) device, and a personal digital assistant (PDA).

The UE 101 can include one or more applications 111a-111n (collectively referred to as applications 111). The applications 111 can be any type of application and can function to achieve aspects of the present disclosure related to selecting specialist physicians to include within a narrow-network healthcare plan.

In one embodiment, one or more of the applications 111 can interface with the platform 103 for selecting specialist physicians to include within a narrow-network healthcare plan. In some embodiments, the platform 103 can be embodied within the UE 101 as one or more of the applications 111.

In one or more embodiments, the UE 101 can be used to administer the peer reviews for determining the reputation score. For example, the UE 101 can be a terminal where a participant of the peer review conducts the review. The UE 101 can also be a computing device that is used to electronically enter a peer review into, for example, the databases 109.

The system 100 further includes electronic health record data providers 107a-107n (collectively referred to as data providers 107). The data providers 107 can provide the electronic health record data discussed above for determining the performance score of a specialist physician, such as a third-party entity other than the healthcare providers that provides the electronic health record data to the healthcare providers. Alternatively, the data providers 107 can be operated, maintained, and controlled by one or more healthcare providers, such as one or more hospitals, medical groups, etc., and can be used to collect and store the electronic health record data. Although shown and described as two separate components within the system 100, in some aspects, the UE 101 and the data provider 107 can be operated, maintained, and controlled by the same entity, as well as embodied within the same device, such as the same computing device. The data providers 107 can also provide the information associated with the peer reviews of the specialist physicians. In one or more embodiments, the data providers 107 can also be the administrators of the peer reviews. Alternatively, the healthcare providers can administer the peer reviews and the information generated from the peer reviews can be submitted to the data providers 107.

The system 100 further includes electronic health record databases 109a-109n (collectively referred to as databases 109). The databases 109 can be used by the healthcare providers and/or third-party data providers 107 to store the electronic health record data used to determine the performance score for a specialist physician. The databases 109 can also store the information associated with the peer reviews of the specialist physicians. Although shown and described as a separate component within the system 100, in some aspects, the data provider 107 and the database 109 can be the same component within the system 100.

The platform 103 performs the functions described herein for selecting specialist physicians to include within a narrow-network healthcare plan. The platform 103 can communicate with one or more components of the system 100, such as the data providers 107 and the databases 109 to collect the electronic health care data and determine the specialist physicians to include within the narrow-network healthcare plan based on the data.

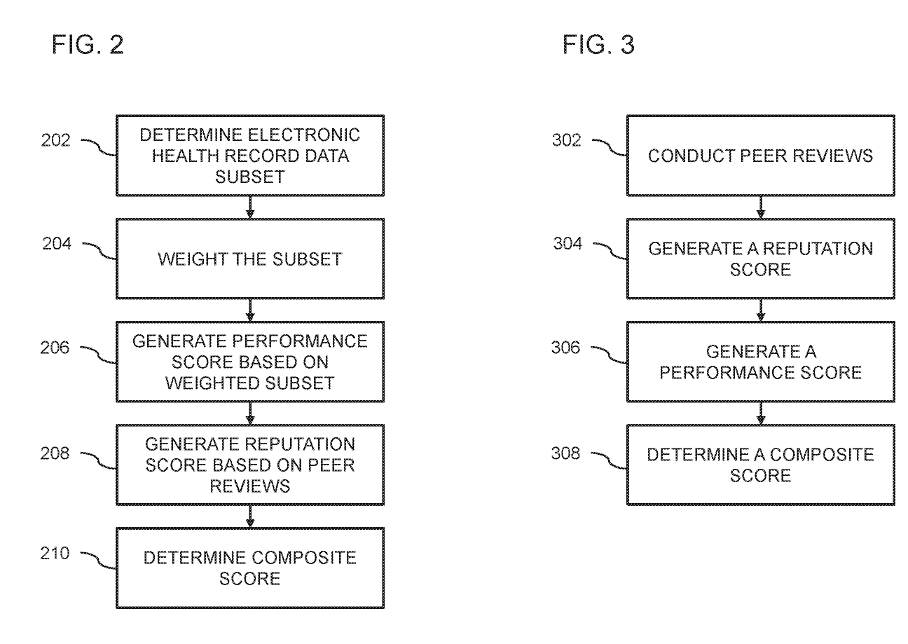

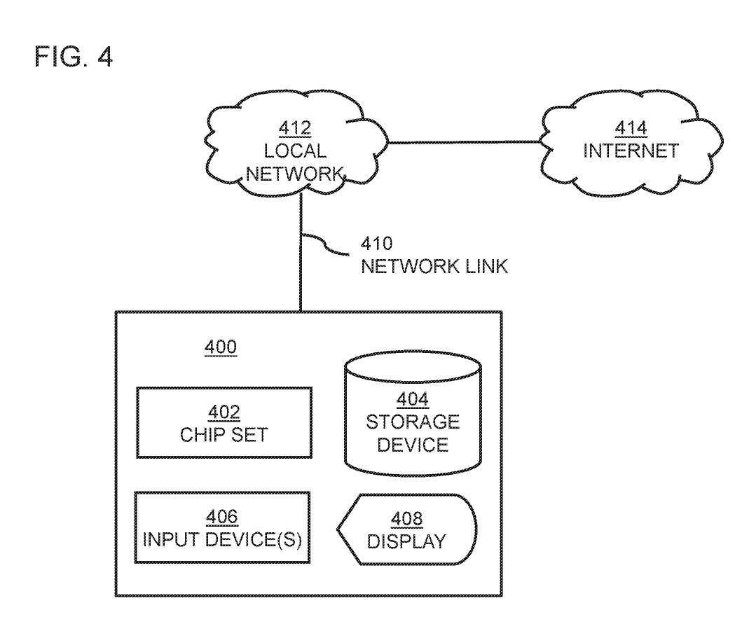

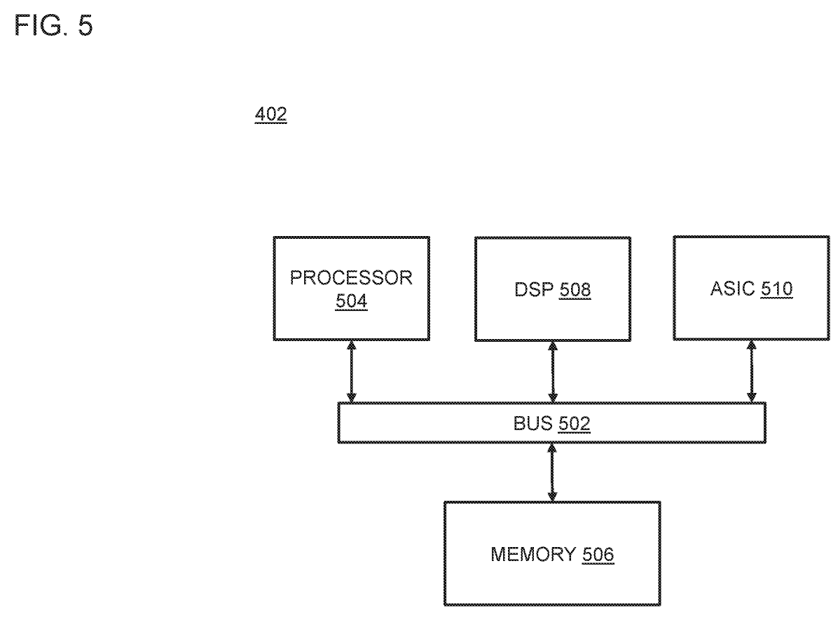

Referring to FIG. 2, FIG. 2 is a flowchart of a process for including one or more specialist physicians in a narrow-network healthcare plan, in accord with aspects of the present disclosure. The process can be performed by the platform 103, which can be embodied in one or more of the hardware illustrated in FIGS. 4 and 5 and described below.

At step 202, a subset of electronic health record data is determined that corresponds to one or more specialist physicians that are being evaluated for inclusion within a narrow-network healthcare plan. The electronic health record data also corresponds to a plurality of patients treated by the one or more specialist physicians and metrics that quantify the quality of care, the efficiency of care, or a combination thereof provided by the one or more specialist physicians.

The one or more specialist physicians can be a subset of physicians associated with a healthcare entity. The subset of the one or more specialist physicians can be based on the type of specialty of the specialist physicians, such as the specialties described above. In some aspects, the subset of the electronic health record data also can be based on the type of specialty, such as different electronic health record data being used for different specialties. For example, the subset of the electronic health record data can be the collected metrics that are the most demonstrative for the quality of care, the efficiency of care, or a combination thereof of the specialty. In some aspects, the subset can be the top six, the top five, the top four, the top three, etc. metrics of the specialty. As discussed above, the healthcare entity can be a hospital, a network of hospitals, a private medical practice, or a combination thereof, or any entity described herein.

At step 204, a weighting is applied to the subset of the electronic health record data. In some aspects, the weighting can be based on the type of specialty such that different weighting is applied for different specialties. In some aspects, the weighting that is applied to the electronic health record data can be based on surveying the participants to determine a weighting value for each metric. In other words, the specialist physicians, general physicians, administrators, etc. can determine what weightings to apply the electronic health record data. Further, the weighting can vary with respect to the specialty of the physician. For example, length of stay can be weighted different for a cardiologist as compared to a neurologist, and both weightings can be based on surveys.

At step 206, the weighted subset of the electronic health record data is processed to generate a performance score for each specialist physician of the one or more specialist physicians. In some aspects, the weighting applies a larger importance to each normalized metric of the subset of the electronic health record data that is directly related to the type of specialty. The processing can be as described above with respect to determining the z-sores and a combination of the calculated z-scores so that the data can be normalized across the one or more specialist physicians.

At step 208, a plurality of peer reviews completed by participants associated with the healthcare entity concerning the one or more specialist physicians are processed to generate a reputation score for each of the specialist physicians. The reputation score quantifies reputations of the specialist physicians among the participants associated with the healthcare entity and peer reviews. The plurality of peer reviews can be based on an appropriateness scale peer review format.

At step 210, the performance score and the reputation score are processed for each of the specialist physicians to determine a composite score for selecting the one or more specialist physicians to include in the narrow-network healthcare plan. As discussed above, there are various ways of determining the composite score based on the performance score and the reputation score. In some aspects, a determination can be made as to whether there is missing electronic health record data for each specialist physician. If there is missing data, the composite score can be determined by accounting for the missing electronic health record data based on the reputation score when determining the composite score. The composite score also can be generated by weighting the performance score equal to the reputation score, weighting the performance score more or less than the reputation score, or not weighting the performance score and the reputation score at all, such as by using the raw values of the two metrics.

After determining the composite score, the score can be used to determine which specialist physicians to include within the narrow-network healthcare plan. There can be a threshold composite score such that, for example, physicians with scores above or below the threshold are included or excluded from the narrow-network healthcare plan. In some aspects, the specialist physicians with the top 5, 10, 20, etc. scores can be included within the narrow-network healthcare plan. Other ways of distinguishing the specialist physician can be used that are based on the composite score.

In one or more embodiments, the process of FIG. 2 can occur once, such as at an initial determination of which specialist physicians to include within in a narrow-network healthcare plan. Thereafter, the process can stop. Alternatively, after the initial determination, the process can repeat one or more times, up to and including continuously, to dynamically update which specialist physicians should be included within the narrow-network healthcare plan. For example, the narrow-network platform 103 can be linked to the data providers 107 and/or the databases 109 to continuously acquire and process new data, such as new electronic health record data. The new electronic health record data can then be taken into consideration for determining updates on the specialist physicians to include within the narrow-network healthcare plan. In one embodiment, the narrow-network platform 103 can generate one or more alerts that provide an indication when a variation exists between the specialist physicians that are included within a narrow-network healthcare plan and the specialist physicians that should be included in the plan based on the new electronic health record data. Accordingly, the narrow-network platform 103 allows for the process of FIG. 2 to run in the background as electronic health record data is collected and then provide notice to healthcare providers regarding updates on specialist physicians to include with a narrow-network healthcare plan.

Referring to FIG. 3, FIG. 3 is a flowchart of a process for modifying a peer review of an individual to appear quantitative without changing an outcome of the peer review, in accord with aspects of the present disclosure. The process can be performed by the narrow-network platform 103, which can be embodied in one or more of the hardware illustrated in FIGS. 4 and 5 and described below.

At step 302, a peer review of an individual is conducted. The individual being reviewed can be a specialist physician at a healthcare entity. The peer review can include surveying a plurality of participants related to the healthcare entity for opinions of the plurality of participants related to the individual. The survey can be formatted as described above. There can be as little as one question or statement by which to review the individual, or more than one question or statement.

At step 304, the peer reviews are processed to generate a reputation score for the individual. The processing can include, for example, summing, averaging, normalizing, etc. the outcomes of the surveys to generate a reputation score.

At step 306, electronic health record data corresponding to treatment of a plurality of patients by the individual can be processed to generate a performance score. The performance score can quantify a quality of care, an efficiency of care, or a combination of both quality and efficiency of care provided to the plurality of patients by the individual. The processing can occur as discussed with respect to Equations 1-4 above. In some aspects, the processing of the electronic health record data can include processing only a subset of the electronic health record data, where the subset is based on the type of specialty of the individual. In some aspects, a weighting can be applied to the subset of the electronic health record data, and the weighting can be based on the type of specialty. The weighting also can be generated from a survey of the plurality of participants. The participants can be, for example, physicians and administrative staff associated with the healthcare entity, or any other individual described herein.

At step 308, the performance score and the reputation score are processed to determine a composite score applicable to the individual and for determining whether to include the individual within a narrow-network healthcare plan. However, generation of the composite score can be performed by weighting the reputation score relative to the performance score such that the determination of whether to include the individual within the narrow-network healthcare plan depends on only the reputation score. Thus, the individual being rated would believe that the composite score is not, for example, merely a popularity contest because of the addition of the performance score generated from the electronic health record data. However, determination of the composite score is conducted such that the performance score does not affect the ultimate outcome.

The processes described herein for selecting specialist physicians to include within a narrow-network healthcare plan may be advantageously implemented via software, hardware, firmware, or a combination thereof. Such exemplary hardware for performing the described functions is detailed below.

FIG. 4 illustrates a computer system 400 upon which the functions of the disclosed aspects can be implemented. Computer system 400 can be programmed (e.g., via computer program code or instructions) to select specialist physicians to include within a narrow-network healthcare plan, as described herein. The computer system 400 includes a chip set 402, which is further described in FIG. 5. Coupled to the chip set 402 is a storage device 404, such as a magnetic disk, optical disk, or flash card, for storing information, including instructions, that persists even when the computer system 400 is turned off or otherwise loses power.

Information, including instructions for selecting specialist physicians to include within a narrow-network healthcare plan, is provided to the chip set 402 from an input device 406, such as a keyboard containing alphanumeric keys operated by a human user, a pointing device, such as a mouse, a trackball, cursor direction keys, or a motion sensor, for controlling a position of a small cursor image. Other external devices coupled to the chip set 402, used primarily for interacting with humans, include a display device 408, such as a cathode ray tube (CRT), a liquid crystal display (LCD), a light emitting diode (LED) display, an organic LED (OLED) display, a plasma screen, or a printer for presenting text or images.

The computer system 400 can be connected to a network link 410 that is connected to a local network 412 to which a variety of external devices with their own processors can be connected, such as the components illustrated in FIG. 1. The network link 410 typically provides information communication using transmission media through one or more networks to other devices that use or process the information, and can be connected to the Internet 414 for distribution of the information over a wider network (e.g., the globe).

The signals transmitted over network link 410 and other networks carry information to and from computer system 400. Computer system 400 can send and receive information, including program code, through the local network 412 and the Internet 414 among others, through network link 410.

FIG. 5 illustrates a chip set or chip 500 upon which aspects of the present disclosure can be implemented. Chip set 500 is programmed to perform the functions as described herein to select specialist physicians to include within a narrow-network healthcare plan. The chip set or chip 500 can be implemented in a single chip or as a single “system on a chip.” It is further contemplated that, in certain embodiments, a separate ASIC would not be used, for example, and that all relevant functions as disclosed herein would be performed by a processor or processors. Chip set or chip 500, or a portion thereof, constitutes a means for performing one or more steps related to selecting specialist physicians to include within a narrow-network healthcare plan.

As shown, the chip set or chip 500 includes a bus 502 for passing information among the components of the chip set 500. A processor 504 has connectivity to the bus 502 to execute instructions and process information stored in, for example, a memory 506. The processor 504 can include one or more processing core(s) (e.g., one, two, four, eight, or greater numbers of processing cores) with each core configured to perform independently. Alternatively, or in addition, the processor 504 can include one or more microprocessors configured in tandem via the bus 502 to enable independent execution of instructions. The processor 504 can also be accompanied with one or more specialized components to perform certain processing functions and tasks such as one or more digital signal processors (DSP) 508, or one or more application-specific integrated circuits (ASIC) 510. In one embodiment, the chip set or chip 500 includes merely one or more processors and some software and/or firmware supporting and/or relating to and/or for the one or more processors.

The processor 504 and accompanying components have connectivity to the memory 506 via the bus 502. The memory 506 can include dynamic memory (e.g., RAM, magnetic disk, writable optical disk, etc.) and/or static memory (e.g., ROM, CD-ROM, etc.) for storing executable instructions that, when executed, perform the functions described herein. The memory 506 also stores the data associated with or generated by the execution of the inventive steps.

The term “computer-readable medium” as used herein refers to any medium that participates in providing information to the processor 504, including instructions for execution. Such a medium may take many forms, including, but not limited to computer-readable storage medium (e.g., non-volatile media, volatile media), and transmission media. Non-transitory media, such as non-volatile media, include, for example, optical or magnetic disks, such as storage device 404. Volatile media include, for example, dynamic memory. Transmission media include, for example, twisted pair cables, coaxial cables, copper wire, fiber optic cables, and carrier waves that travel through space without wires or cables, such as acoustic waves and electromagnetic waves, including radio, optical and infrared waves. Signals include man-made transient variations in amplitude, frequency, phase, polarization or other physical properties transmitted through the transmission media. Common forms of computer-readable media include, for example, a floppy disk, a flexible disk, hard disk, magnetic tape, any other magnetic medium, a CD-ROM, CDRW, DVD, any other optical medium, punch cards, paper tape, optical mark sheets, any other physical medium with patterns of holes or other optically recognizable indicia, a RAM, a PROM, an EPROM, a FLASH-EPROM, an EEPROM, a flash memory, any other memory chip or cartridge, a carrier wave, or any other medium from which a computer can read. The term computer-readable storage medium is used herein to refer to any computer-readable medium except transmission media.

While the present disclosure relates discloses one or more particular embodiments, those skilled in the art will recognize that many changes may be made thereto without departing from the spirit and scope of the present invention. Each of these embodiment(s) and obvious variations thereof is contemplated as falling within the spirit and scope of the invention. It is also contemplated that additional embodiments according to aspects of the present invention may combine any number of features from any of the embodiments described herein.

What is claimed is:

1. A method for including one or more specialist physicians in a narrow-network healthcare plan, comprising:
determining a subset of electronic health record data corresponding to the one or more specialist physicians, the one or more specialist physicians being a subset of physicians associated with a healthcare entity based on a type of specialty, and the subset of the electronic health record data being based on the type of specialty;
applying a weighting to the subset of the electronic health record data, the weighting being based on the type of specialty;
processing the weighted subset of the electronic health record data to generate a performance score for each specialist physician of the one or more specialist physicians;
processing a plurality of peer reviews completed by participants associated with the healthcare entity concerning the one or more specialist physicians to generate a reputation score for each specialist physician of the one or more specialist physicians, the reputation score quantifying reputations of the one or more specialist physicians among the participants associated with the healthcare entity; and
processing the performance score and the reputation score for each specialist physician of the plurality of specialist physicians to determine a composite score for selecting which of the one or more specialist physicians to include in the narrow-network healthcare plan.

2. The method of claim 1, further comprising:
normalizing each metric within the subset of the electronic health record data across the one or more specialist physicians,
wherein the weighting applies a larger importance to each normalized metric of the subset of the electronic health record data that is directly related to the type of specialty.

3. The method of claim 1, wherein the electronic health record data corresponds to a plurality of patients treated by the one or more specialist physicians.

4. The method of claim 1, wherein the electronic health record data includes a plurality of metrics that relate to the quality of care, the efficiency of care, or a combination thereof provided to the plurality of patients.

5. The method of claim 4, further comprising:
surveying the participants to determine a weighting value for each metric of the plurality of metrics within the electronic health record data,
wherein the applying of the weighting is based on the weighting values for the plurality of metrics.

6. The method of claim 4, further comprising:
determining missing electronic health record data for each specialist physician among the one or more specialist physicians; and
accounting for the missing electronic health record data based on the reputation score when determining the composite score.

7. The method of claim 1, further comprising:
weighting the performance score relative to the reputation score for each specialist physician for the one or more specialist physician in determining the composite score.

8. A method of modifying a peer review of an individual to appear quantitative without changing an outcome of the peer review, comprising:
conducting the peer review of the individual, the individual being a specialist physician at a healthcare entity, the peer review including surveying a plurality of participants related to the healthcare entity for opinions of the plurality of participants related to the individual;
processing the peer review to generate a reputation score for the individual;
processing electronic health record data corresponding to treatment of a plurality of patients by the individual to generate a performance score, the performance score quantifying a quality of care, an efficiency of care, or a combination thereof provided to the plurality of patients by the individual; and
processing the performance score and the reputation score to determine a composite score applicable to the individual and for determining whether to include the individual within a narrow-network healthcare plan,
wherein the reputation score is weighted relative to the performance score such that the determination of whether to include the individual within the narrow-network healthcare plan depends on only the reputation score.

9. The method of claim 8, wherein the processing of the electronic health record data comprises processing a subset of the electronic health record data, the subset being based on the type of specialty of the individual.

10. The method of claim 8, further comprising:
applying a weighting to the subset of the electronic health record data, the weighting being based on the type of specialty,
wherein the weighting is generated from a survey of the plurality of participants.

11. A system for including one or more specialist physicians in a narrow-network healthcare plan, comprising:
one or more databases storing electronic health record data; and
logic circuitry configured to:
determine a subset of the electronic health record data corresponding to the one or more specialist physicians, the one or more specialist physicians being a subset of physicians associated with a healthcare entity based on a type of specialty, and the subset of the electronic health record data being based on the type of specialty;
apply a weighting to the subset of the electronic health record data, the weighting being based on the type of specialty;
generate a performance score for each specialist physician of the one or more specialist physicians based on the weighted subset of the electronic health record data;
generate a reputation score for each specialist physician of the one or more specialist physicians based on a plurality of peer reviews completed by participants associated with the healthcare entity concerning the one or more specialist physicians, the reputation score quantifying reputations of the one or more specialist physicians among the participants associated with the healthcare entity; and
determine a composite score for selecting the one or more specialist physicians to include in the narrow-network healthcare plan based on the performance score and the reputation score for each specialist physician of the plurality of specialist physicians.

12. The system of claim 11, the logic circuitry being further configured to:
normalize each metric within the subset of the electronic health record data across the one or more specialist physicians,
wherein the weighting applies a larger importance to each normalized metric of the subset of the electronic health record data that is directly related to the type of specialty.

13. The system of claim 11, wherein the electronic health record data corresponds to a plurality of patients treated by the one or more specialist physicians.

14. The system of claim 11, wherein the electronic health record data includes a plurality of metrics that relate to the quality of care, the efficiency of care, or a combination thereof provided to the plurality of patients.

15. The system of claim 14, the logic circuitry being further configured to:
survey the participants to determine a weighting value for each metric of the plurality of metrics within the electronic health record data,
wherein the applying of the weighting is based on the weighting values for the plurality of metrics.

16. The system of claim 14, the logic circuitry being further configured to:
determine missing electronic health record data for each specialist physician among the one or more specialist physicians; and
account for the missing electronic health record data based on the reputation score when determining the composite score.

17. The system of claim 11, the logic circuitry being further configured to:
weight the performance score relative to the reputation score for each specialist physician for the one or more specialist physician in determining the composite score.

18. A system for modifying a peer review of an individual to appear quantitative without changing an outcome of the peer review, comprising:
a terminal for receiving the peer review, the peer review including a survey of a plurality of participants related to the healthcare entity for opinions of the plurality of participants related to the individual; and
logic circuitry configured to:
generate a reputation score for the individual based on the peer review;
generate a performance score based on electronic health record data corresponding to treatment of a plurality of patients by the individual, the performance score quantifying a quality of care, an efficiency of care, or a combination thereof provided to the plurality of patients by the individual; and
determine a composite score applicable to the individual based on the performance score and the reputation score for determining whether to include the individual within a narrow-network healthcare plan,
wherein the reputation score is weighted relative to the performance score such that the determination of whether to include the individual within the narrow-network healthcare plan depends on only the reputation score.

19. The system of claim 18, wherein the generation of the performance score comprises processing a subset of the electronic health record data, the subset being based on the type of specialty of the individual.

20. The system of claim 18, further comprising:
apply a weighting to the subset of the electronic health record data, the weighting being based on the type of specialty,
wherein the weighting is generated from a survey of the plurality of participants.
